(19) United States
(12) Patent Application Publication
(10) Pub. No.: US 2010/0310755 A1
(43) Pub. Date: Dec. 9, 2010

(54) ULTRA-SENSITIVE DETECTION OF FAINT FINGERPRINTS ON ROUGH SURFACES AND CORRESPONDING FINGERPRINT DETECTION KIT

(75) Inventor: Amir J. Attar, Raleigh, NC (US)

Correspondence Address: INTELLECTUAL PROPERTY / TECHNOLOGY LAW, PO BOX 14329, RESEARCH TRIANGLE PARK, NC, 27709, US

(73) Assignee: APPEALING PRODUCTS, INC., Raleigh, NC (US)

(21) Appl. No.: 12/478,640

(22) Filed: Jun. 4, 2009

(51) Int. Cl. A61B 5/117

(52) U.S. Cl. 427/1; 118/620

(57) ABSTRACT

An ultra-sensitive method for visualizing latent fingerprints using a chemical multiplier to amplify even very faint prints on rough or otherwise difficult surfaces, involving a chemical free radical reaction which produces millions of identifiable molecules from each adsorbed initiator molecule and thereby greatly increases the detection sensitivity. A corresponding fingerprint detection kit may be utilized to carry out such method, in which the kit includes a free radical chain reaction initiator, and a reagent that can react via the free radical chain reaction in the presence of the initiator and form colored, fluorescent, polymeric, IR- or UV-absorbing molecules that are effective for visualization of the fingerprint.

### BACKGROUND OF THE INVENTION

One of the most difficult tasks in forensic investigation is “lifting” old, partially deteriorated, latent fingerprints. This task is difficult even when the surface is smooth, but is extremely difficult when the surface is rough, such as course ceramic, concrete or rough paper. Being able to lift old and degraded fingerprints is an extremely important forensic tool in fighting crime, as well as terrorism. In consequence, technology for lifting fingerprints has been studied very extensively.

Fingerprint residues consist of moisture, salt, fat, amino acids, urea and other materials. Classical visualization of fingerprints involves spreading fine powder, often carbon black or pollen, on the prints. The powder concentrates selectively in the fingerprint residue and thus allows visualization of the latent prints. Typically, the visualized fingerprint is photographed or digitally scanned for record keeping. Numerous powders and formulations are available commercially (see, e.g., http://www.sirchie.com/, the website of Sirchie Fingerprints Laboratories, Wake Forest, N.C., one of the biggest suppliers of such materials) and many fingerprint developing reagents are continually added.

Different chemical reagents have been developed that extend the capacity to lift latent fingerprints beyond that provided by fine carbon black or pollen-based powders. Typically, a chemical reagent is sprayed on the latent prints. The reagent reacts selectively with one of the components of the latent prints and produces a visual result such as a color change. Many of these methods are described in the commercial catalogs of forensic hardware suppliers (see, for example, http://www.sirchie.com/).

Reagents have been developed that react chemically with every component of the fingerprint residue. For example, ninhydrin reacts with the amino acids to form a purple color. Silver nitrate solution produces silver chloride by reacting with the sodium chloride in sweat and later decomposes to form black metallic silver that allows visualization of the prints. Vapors of cyanoacrylate react with the moisture of the prints, polymerize and form a visible print corresponding to the ridges. Iodine vapors dissolve in the fats and the lipids of the prints and form visible color. Adding a zinc solution to the ninhydrin prints yields a product that fluoresces in UV light and increases the detection sensitivity.

An extremely expensive yet sensitive reagent that reacts with proteins and amino acids is 1,8 diazafluoren-9-one, DFO, which produces a much stronger fluorescence than ninhydrin. Nevertheless, the reaction products of amino acid residues with ninhydrin or DFO are stoichiometric and therefore have only limited sensitivity.

Many additional reagents have been tried by different groups to achieve high sensitivity, notably the organic synthesis group of Prof. M. Joullie at the University of Pennsylvania, who worked closely with the U.S. Secret Service on synthesizing various reagents (Ramotowski, R., Cantu, A. A., Joulliè, M. M., and Petrovskaia, O. 1,2-indanediones: A preliminary evaluation of a new class of amino acid visualizing compounds, Fingerprint Whorld (1997) 23(90): 131-140).

A very substantial amount of work was done by the Israeli Police Department, notably by Dr. Almog and coworkers (Almog, J., Bahar, E., Dayan, S., Frank, A., Khodzhaev, O., Lidor, R., Razen, S., Springer, E., Varkony, H., and Wiesner, S. Latent fingerprint visualization by IND and related compounds: Preliminary results, Journal of Forensic Sciences (1999) 44(1):114-118; and Wiesner, S., Almog, J., Sasson, Y., and Springer, E. Chemical development of latent fingerprints: IND has come of age, Journal of Forensic Sciences (2001) 46(5):1082-1084.

Another important group which has studied this subject is the Forensic Science Research Unit at the Chemistry Dept. of the Australian National University (Stoilovic, M. et. al, Forensic Science International, 24 (1984) p. 279-284 ; and Warrener, R. N. et. al IBID, 23 (1983), p. 179-188).

The important conclusion from reviewing all of the above-referenced literature and trying some of the proposed chemistries is that detection of latent fingerprints was enhanced, but only to the extent that the reagent used formed more intense color than previously described reagents. The reaction was still limited to the stoichiometric ratio of 1-3 colored molecules per amino acid molecule.

### SUMMARY OF THE INVENTION

The present invention relates to the ultra-sensitive detection of faint fingerprints on rough or otherwise difficult surfaces and a corresponding fingerprint detection kit.

The invention in one aspect relates to a method useful in visualizing fingerprints, comprising contacting a surface having a potential visualizable fingerprint thereon with a free radical chain reaction initiator, contacting the surface with a reagent capable of reacting via the free radical chain reaction in the presence of the initiator to form a colored, fluorescent, polymeric, infrared radiation-absorbing, or ultraviolet radiation-absorbing reaction product for at least partial visualization of a fingerprint when present on said surface.

In a further aspect, the invention relates to a method for visualization of fingerprints, comprising:

exposing a surface suspected of having one or more fingerprints on it to a vapor, mist or solution of chemical material capable of initiating a free radical chain reaction; and

exposing the surface to a vapor, mist or solution of a reagent that can react via the free radical chain reaction and form colored, fluorescent, polymeric, IR or UV-absorbing molecules,

whereby the free radical chain reaction is effected to at least partially visualize the fingerprints.

In a further aspect of the method broadly described above, the free radical chain reaction is initiated using thermal heating, UV or other radiation, it being noted that in many cases ambient temperature or ambient light is sufficient to initiate the chain reaction.

In another aspect, the invention relates to a kit for visualizing fingerprints which includes a free radical chain reaction initiator, a reagent that can react via the free radical chain reaction in the presence of the initiator and form colored, fluorescent, polymeric, IR or UV-absorbing molecules in the presence of a fingerprint, optionally with additional components such as containers and/or applicators for the initiator and reagent components, written indicia such as instruction sheets or instruction booklets, heaters, radiation sources, recorders, e.g., cameras, tablets, PDAs, etc., for recording the results of the use of the kit.

Other aspects, features and embodiments of the invention will be more fully apparent from the ensuing disclosure and appended claims.

### BRIEF DESCRIPTION OF THE DRAWINGS

FIG. 1 shows an original surface of rough paper after imprinting on it a thumb print.

FIG. 2 shows the same surface of FIG. 1 after fuming it with small amount of an initiator.

FIG. 3 shows the same surface of FIG. 2 after spraying it with a chromophore that can be oxidized by a free radicals chain reaction, resulting in a reaction that produced a clearly visible fingerprint pattern from the latent print.

### DETAILED DESCRIPTION OF THE INVENTION, AND PREFERRED EMBODIMENTS THEREOF

The present invention relates to a novel and extremely sensitive class of chemistries that can be used to visualize faint fingerprints on a variety of surfaces and a kit that can be used to implement the chemistries in lab and field conditions.

The invention in one aspect relates to a method useful in visualizing fingerprints, comprising contacting a surface having a potential visualizable fingerprint thereon with a free radical chain reaction initiator, contacting the surface with a reagent capable of reacting via the free radical chain reaction in the presence of the initiator to form a colored, fluorescent, polymeric, infrared radiation-absorbing, or ultraviolet radiation-absorbing reaction product for at least partial visualization of a fingerprint when present on said surface.

In a further aspect, the invention relates to a method for visualization of fingerprints, comprising:

exposing a surface suspected of having one or more fingerprints on it to a vapor, mist or solution of chemical material capable of initiating a free radical chain reaction; and

exposing the surface to a vapor, mist or solution of a reagent that can react via the free radical chain reaction and form colored, fluorescent, polymeric, IR or UV-absorbing molecules,

whereby the free radical chain reaction is effected to at least partially visualize the fingerprints.

Such free radical chain reaction can be initiated using thermal heating, UV or other radiation, if ambient temperature or ambient light is not sufficient to initiate the chain reaction for the specific initiators and reagents employed in specific applications.

In another aspect, the invention relates to a kit for visualizing fingerprints which includes a free radical chain reaction initiator, and a reagent that can react via the free radical chain reaction in the presence of the initiator and form colored, fluorescent, polymeric, IR or UV-absorbing molecules effective to at least partially visualize a fingerprint.

The kit may also include suitable containers and/or applicators for the initiator and reagent components, UV sources, IR light sources, or lamps providing the specific radiation needed for initiation of the free radical chain reaction, heater devices for thermal activation of the free radical chain reaction, printed indicia such as instruction sheets for directing the use of the other kit components, etc.

The common denominator of all the chemistries involves the use of a free radical chain initiator that adsorbs selectively on the remains of the fingerprint. A second spray of a material, which can react via a free radical chain reaction and form color, follows the initiator. The initiator initiates a free radical chain reaction which forms millions of colored molecules per each successful chain initiated and thus produces a clear and visible image of the fingerprint. This reaction is very different than conventional visualization techniques since all the previously reported chemistries produce only one to three colored molecules based on the reaction stoichiometry.

Some of the free radical chain reactions useful in the broad practice of the present invention may also involve air or oxygen. Some initiators useful in the practice of the present invention require irradiating the surface with ultra-violet radiation after adding the free radical chain reaction-effecting reagents. The invention may be practiced using a variety of specific chemicals, within the skill of the art based on the disclosure herein, without undue experimentation.

Application of the initiator or the reagent can be done by fuming vapors onto the surface or by spraying it with a fine liquid mist of the solution, or in other manner contacting the initiator and reagent with the substrate bearing the latent fingerprint in a fluid or other suitable form.

Examples of initiators are iodine vapors, bromine vapors, chlorine gas, peroxides such as hydrogen peroxide or organic peroxide such as t-butyl-peroxides, benzoyl peroxide, (BP), perbenzoic acid or its derivatives such as m-bromo-perbenzoic acid, hydroperoxides such as t-butyl hydroperoxide, (tBHP), cumyl hydroperoxide, etc. and certain nitrogen compounds such as azo-bis-iso-butyro-nitrile (AIBN). The particular listing of possible initiators is presented for demonstration purposes and is not meant to limit or restrict the scope of the materials that may be used in this invention. As used herein, the term “gas” is intended to be broadly construed to include gas as well as vapor.

Three useful classes of compounds that may be used as reagents, which react in a free radical chain to form colored compounds are:

1. Aromatic amines or polyamines. Examples of such compounds include tetra-methyl-benzidine, (TMB), tetra-methoxy-benzidine, benzidine, di-methoxy-benzidine, etc.

2. Compounds that utilize hydroxylated, conjugated aromatic compounds such as alizarins.

3. Compounds that form new conjugated aromatic rings or where rings open as a result of the reaction, thus resulting in a change in the UV absorption of the compound and/or in its fluorescence or other radiation-interactive properties.

The particular listing of possible chromophores is presented for demonstration purpose and is not meant to limit or restrict the scope of the materials that may be used in this invention.

A lab or field kit for detecting fingerprints is also contemplated within the broad scope of the present invention. In one embodiment, the kit includes kit components for accomplishing four goals:

1. Gently covering the suspect surface with vapors or a fine mist of a compound capable of initiating a free radical chain reaction by thermal mediation and/or via the use of UV light or other radiation;

2. Gently covering the suspect surface with vapors or a fine mist of a compound capable of reacting by a free radical chain reaction and forming colored or fluorescent compounds in the visible or UV range;

3. UV light source for short or long UV light for initiating the free radical chain reaction and/or for visualizing the fingerprints formed; and

4. A camera or other recorder, for recording the results of the visualization of the fingerprints. The results can be fingerprints in the visible, UV or IR range and the recording means must be capable of accommodating these results.

Chemical Amplification Using Free Radical Chains.

Basic free radical chain sequences include three types of reactions:

A. Initiation Reactions, where more free radicals are formed than were present in the starting materials, e.g., as produced by the decomposition of a peroxide:

In₂→2In. Rate constant: kᵢ (1)

ROOR→2RO. (2)

(C₆H₅C(O)O)₂→2C₆h₅C(O)O. (3)

or by the thermal decomposition of acetaldehyde:

CH₃C(O)H→.CH₃+.CHO (4)

(It is to be appreciated that for ease of reference, the notation CH₃. is used frequently instead of .CH₃; these expressions are intended to be treated as equivalents of one another).

B. Propagation Reactions, where the number of free radicals does not change in the reaction, e.g., the thermal decomposition of acetaldehyde:

RnH+R.→RH+Rn. Limiting rate constant for the propagation: kₚ (5)

Rn.→R.+P₂ (6)

CH₃C(O)H+CH₃.→CH₃C(O).+CH₄ (7)

CH₃C(O).→CH₃.+CO (8)

Note that the propagation step frequently involves two or more reactions. In addition, there are several radical transfer reactions that do not change the number of free radicals, yet are not true propagation steps. This occurs, for example, when the radicals formed are stable thermodynamically.

C. Termination Reactions, in which free radicals combine to form non-radical products. These reactions reduce the number of free radicals in the system, for example, in the recombination of methyl radicals.

2R.→R₂ Rate constant: kₜ (9)

2CH₃.→C₂H₆ (Presumed the dominant termination reaction) (10)

There are many textbooks and papers related to the kinetic analysis of various types of free radical-related chemical reactions, and no attempt will be made to exhaustively detail them herein. Two important references which summarize a significant portion of the older literature are by Denisov and coworkers (Denisov, E. T., Denisova, T. G., and Pokidova, T. S., “Handbook of Free Radicals Initiators”, Wiley-Interscience, N.Y., (2003); and Emanuel, N. M, Denisov, E. T. and Maizus, Z. K. “Liquid Phase Oxidation of Hydrocarbons”. Plenum Press, N.Y. (1967)).

Kinetics Considerations

Analysis of the simplest sequence of free radical chain reaction, using the conventional assumptions relative to the steady state approximation in the differential equations, yields:

dC/dt=˜kₚ*[RH]*sqrt(2*kᵢ[In₂]/kₜ) (11)

wherein C is the measure of the result of the chain reaction, such as a colored or a fluorescent product. Equation 11 shows that the rate of color formation, dC/dt depends linearly on the chromophore concentration, [RH], and on the square root of the initiator concentration, [In₂], provided that a large excess of RH is available relative to In₂. The total color formed due to exposure to a dose, [In₂], of analyte is approximately:

C=2*kₚ*[RH]*sqrt(2/kᵢ/kₜ)*sqrt(In₂) (12)

This equation presents two very important facts:

1. The amount of color formed for a given amount of the initiator, In₂, can be much larger then the normal stoichiometric values obtained using reactions with simple stoichiometry (that yield 1-3 colored molecules per reaction); and

2. More color is formed when the concentration of In2 is small (per unit In2), than when In2 is larger. In other words, the detector is more sensitive at lower In2,assuming that the analyte to be detected plays the role of In2. This can be readily seen from the following equation:

dC/d(In₂)=kₚ[RH]sqrt(2/kᵢ/kₜ)/sqrt(In₂) (13)

Special Case: Oxidation

One of the most important types of free radical chain reaction is the oxidation of organic compounds. Oxidation essentially follows the same sequence of steps described previously, but it involves several additional twists. Attention will be focused for a moment only on oxidation with oxygen or air.

Most oxidation reactions of hydrocarbons with air or oxygen are autocatalytic in character. In other words, one or more of the oxidation products can dramatically enhance the apparent conversion rate. A typical oxidation sequence will include some of the following reactions (some other typical reactions were not included for simplicity):

In₂→2In. Initiation (14)

In.+RH→InH+R. Propagation (15)

R.+O₂→ROO. Propagation Fast with E˜0 KJ (16)

ROO.+RH→ROOH+R. Propagation (17)

ROOH→RO.+HO. Initiation (18)

RO.+RH→ROH+R. Propagation (19)

HO.+RH→H₂O+R. Propagation (20)

2RO.→ROOR Termination (21)

Some of the remarkable features which the above sequence demonstrates are:

A. The autocatalytic effect of initiation due to the decomposition of reaction products such as the hydroperoxide ROOH. Many other initiation steps occur in most oxidation reactions. Initiation of reactions by such mechanisms is critical in many common processes such as combustion and explosion.

B. The incorporation of air oxygen into intermediates and reaction products. This process will occur in all open-container oxidations and is of particular significance where the reactions are conducted in nano layers and thin films. In these reactions, the ratio of surface area to volume is very large.

C. Since the totality of the oxidation is an exothermic process, the reaction rate will self-accelerate due to the steep dependence of many critical steps on the temperature. The Arrhenius equation shows this dependence:

k=kₒ*exp(−E/(RT)) (22)

where E is the activation energy and k₀ is the pre-exponential factor.

Again, the totality of the discussion shows that a very effective chemical multiplier, which forms numerous colored molecules per successful reaction, can be obtained using a free radical chain reaction, particularly if the chain involves oxidation and the colored reaction products are formed by the oxidation process.

The operative conclusion from this discussion is that utilizing free radical chains can create a very large number of colored, fluorescent or UV absorbing molecules on the ridges which originally absorbed the initiator. The selection of the particular initiator is suitably based on the selectivity of the absorption of the initiator in the ridge material relative to its absorption in the base substrate. The chromogenic reagent is appropriately selected based on the initiator.

Thus, the invention provides in various aspects a method and fingerprint detection kit for the ultra-sensitive detection of faint fingerprints on rough surfaces and a corresponding fingerprint detection kit. As used herein, the term “fingerprints” is intended to be broadly inclusive of patterned residues resulting from contact of a body part with a material surface on which such patterned residues can be visualized by the method of the invention. Such patterned residues include fingerprints per se, palm prints, footprints, toe prints, and the like.

The method and kit of the invention are thus useful in human forensics, tracking, transportation passenger screening, criminal investigations, etc., as well as in a variety of corresponding and other non-human animal and veterinary applications.

The features and advantages of the invention are more fully shown by the following illustrative examples, which are intended to be only illustrative, and not limiting in any way as regards the scope and features of the present invention.

### EXAMPLE 1

Fume the suspect surface with iodine vapors from an iodine tube for 2 minutes and immediately spray the surface with a fine mist of saturated TMB solution in 70% isopropyl alcohol (IPA). The fingerprints appear as blue lines in a few seconds. The fingerprints appear in red if a solution of 2,2′-di-methoxy-benzidine, (2,2′ DMB), is used instead of the TMB.

### EXAMPLE 2

As in Example 1, but using bromine vapors in a fuming chamber instead of the iodine vapors.

### EXAMPLE 3

As in Example 1, but spraying the surface with a solution of 2% tBHP in 70% IPA. Let the IPA dry for a few minutes and spray it again with a saturated solution of TMB in 70% IPA. The fingerprints appear as blue lines in a few minutes. The fingerprints appear in red if a solution of 2,2′-di-methoxy-benzidine, (2,2′ DMB), is used instead of the TMB. Irradiating the surface with a UV lamp makes the prints appear quicker.

### EXAMPLE 4

As in Example 3, but spraying the surface with a solution of 2% BP in 70% IPA. Let the IPA dry for a few minutes and spray it again with a saturated solution of TMB in 70% IPA. The fingerprints appear as blue lines in a few minutes. The fingerprints appear in red if a solution of 2,2′-di-methoxy-benzidine, (2,2′ DMB), is used instead of the TMB. Irradiating the surface with a UV lamp makes the prints appear quicker.

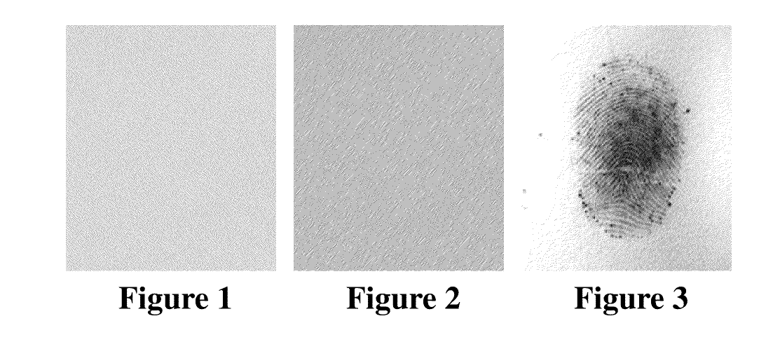

FIGS. 1-3 show the application of the present invention to visualize a latent fingerprint. FIG. 1 shows an original surface of rough paper after imprinting on it a thumb print. FIG. 2 shows the same surface of FIG. 1 after fuming it with small amount of an initiator. FIG. 3 shows the same surface of FIG. 2 after spraying it with a chromophore that can be oxidized by a free radicals chain reaction, resulting in a reaction that produced a clearly visible fingerprint pattern from the latent print.

While the invention has been has been described herein in reference to specific aspects, features and illustrative embodiments of the invention, it will be appreciated that the utility of the invention is not thus limited, but rather extends to and encompasses numerous other variations, modifications and alternative embodiments, as will suggest themselves to those of ordinary skill in the field of the present invention, based on the disclosure herein. Correspondingly, the invention as hereinafter claimed is intended to be broadly construed and interpreted, as including all such variations, modifications and alternative embodiments, within its spirit and scope.

What is claimed is:

1. A method useful in visualizing fingerprints, comprising contacting a surface having a potential visualizable fingerprint thereon with a free radical chain reaction initiator, contacting the surface with a reagent capable of reacting via the free radical chain reaction in the presence of the initiator to form a colored, fluorescent, polymeric, infrared radiation-absorbing, or ultraviolet radiation-absorbing reaction product for at least partial visualization of a fingerprint when present on said surface.

2. The method of claim 1 where the initiator comprises a gaseous compound selected from the group consisting of iodine, bromine, chlorine, nitrogen dioxide, hydrogen peroxide and ozone.

3. The method of claim 1, wherein the initiator comprises a solution of a compound selected from the group consisting of organic peroxides, hydroperoxides, peroxy acids, diazo and azo compounds.

4. The method of claim 1, wherein the initiator is applied as a spray or a mist of a solution of the initiator.

5. The method of claim 1, wherein the reagent comprises a solution of a chromogenic compound selected from the group consisting of organic compounds that include aromatic amines with single or multiple rings and/or single or multiple amine groups.

6. The method of claim 1, wherein the reagent comprises a solution of a chromogenic compound selected from the group consisting of 3,3′-dimethoxybenzidine, benzidine, 4-amino-1-naphthalene sulfonic acid sodium salt, N,N′-diphenylbenzidine, o-Tolidine and 3,3′-dimethoxybenzidine dihydrochloride.

7. The method of claim 1, wherein the reagent comprises a solution of a monomer with an olefinic bond that can be polymerized by a free radical chain reaction.

8. The method of claim 1, wherein initiation of the free radical chain reaction is conducted using radiation selected from the group consisting of visible, IR and UV light.

9. The method of claim 1, wherein initiation of the free radical chain reaction is conducted using heating of the surface with hot air and/or infrared radiation.

10. The method of claim 1, further comprising recording said fingerprint using a camera operative to record images in the visible, infrared or ultraviolet range.

11. A method for visualization of fingerprints, comprising:
exposing a surface suspected of having one or more fingerprints on it, to a vapor, mist or solution of chemical material capable of initiating a free radical chain reaction; and
exposing the surface to a vapor, mist or solution of a reagent that can react via the free radical chain reaction and form colored, fluorescent, polymeric, IR or UV-absorbing molecules,
whereby the free radical chain reaction is effected to at least partially visualize the fingerprints.

12. The method of claim 11, wherein the free radical chain reaction is effected with use of thermal heating, ultraviolet radiation exposure, or a combination thereof.

13. The method of claim 11, wherein the free radical chain reaction is effected under ambient temperature and light conditions.

14. A kit for visualizing fingerprints, comprising a free radical chain reaction initiator, and a reagent that can react via the free radical chain reaction in the presence of the initiator and form colored, fluorescent, polymeric, IR or UV-absorbing molecules in the presence of a fingerprint.

15. The kit of claim 14, further comprising written instructions for carrying out a method of visualizing fingerprints.

16. The kit of claim 14, further comprising containers for containing the free radical chain reaction initiator, and the reagent.

17. The kit of claim 14, further comprising a recorder for recording results of the use of the kit.

18. The kit of claim 17, wherein the recorder comprises a camera for photographing visualized fingerprints.

19. The kit of claim 14, further comprising applicators for the initiator and/or reagent components.

20. The kit of claim 14, further comprising a radiation and/or heat source.
